(19) United States
(12) Patent Application Publication
(10) Pub. No.: US 2005/0272686 A1
(43) Pub. Date: Dec. 8, 2005

(54) Nucleotide based medicament and method of use for treatment of conditions in humans

(76) Inventors: Alfred J. Dentone, Lodi, CA (US); Charles L. Schel, Ormond Beach, FL (US); John Whitehead, Preston (GB); Peter Koppel, Rheinfelden (CH)

Correspondence Address: JOHN P. O'BANION;O'BANION & RITCHEY LLP, 400 CAPITOL MALL SUITE 1550, SACRAMENTO, CA, 95814, US

(21) Appl. No.: 11/133,143

(22) Filed: May 18, 2005

Related U.S. Application Data
(63) Continuation of application No. PCT/US03/37295, filed on Nov. 21, 2003.
(60) Provisional application No. 60/428,321, filed on Nov. 21, 2002.
(60) Provisional application No. 60/441,205, filed on Jan. 17, 2003.

(51) Int. Cl. A61K 31/7072; A61K 31/7076

(52) U.S. Cl. 514/45; 514/49

(57) ABSTRACT

A dietary supplement comprising nucleotides in a yeast carrier alone or in combination with antioxidants, amino acids and vitamins is provided that is beneficially used for the treatment of irritable bowel syndrome, chronic diarrhea and cold or flu symptoms as well as for modulating serum HDL and LDL cholesterol levels. Dosing protocols for treatment of these conditions are provided.

### CROSS-REFERENCE TO RELATED APPLICATIONS

This application claims priority from, and is a 35 U.S.C. § 111(a) continuation of, co-pending PCT international application serial number PCT/US03/37295 filed on Nov. 21, 2003, which designates the U.S., incorporated herein by reference in its entirety, which claims priority from U.S. provisional application Ser. No. filed 60/428,321 on Nov. 21, 2002, incorporated herein by reference in its entirety, and which claims priority from U.S. provisional application Ser. No. 60/441,205 filed on Jan. 17, 2003, incorporated herein by reference in its entirety. Priority is claimed to each of the foregoing applications.

The foregoing PCT international application was published as International Publication No. WO 2004/047737 A3 on Jun. 10, 2004, incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

This invention pertains generally to pharmaceutical compositions and methods for treatment of humans, and more particularly to methods for treating chronic diarrhea and irritable bowel syndrome, abnormal cholesterol levels and bolstering the immune system of a patient through the use of a nucleotide based medicament and method of use.

2. Background of the Art

The use of naturally occurring plant extracts and other organic materials for specific medical treatments has increased dramatically in recent years. Some natural plant extracts have been shown to have activity as therapeutic or preventive agents when taken as dietary supplements in humans or animals. Investigation of the pharmaceutical properties of the traditional medicines of the indigenous peoples of many countries as well as newly discovered plants have produced significant medical treatments for diseases.

Many dietary supplements effectively treat the symptoms of a disease by stimulating or supporting, for example, the normal immune, nervous, endocrine or vascular systems of the body. Physical ailments may create deficiencies or interfere with the often complex and interrelated subsystems producing characteristic symptoms and chronic conditions.

The human body must obtain some nutrients from the diet for proper function. It has been seen that insufficiencies or excesses in the diet can result in disrupted systems and unwanted symptoms. Dietary supplements of vitamins and therapeutic agents and the like to support the body are well known in the art.

Deoxyribonucleic acid (DNA) and Ribonucleic acid (RNA) comprise the fundamental genetic material of a cell and are comprised of subunits called nucleotides. A nucleotide generally consists of a base, a sugar and a phosphate group. The bases found in DNA are the pyrimidines cytosine and thymidine and the purines adenine and guanine. The bases found in RNA are the purines adenine and guanine and the pyrimidines cytosine and uracil. The purine or pyrimidine bases may be coupled to a deoxyribose sugar or a ribose sugar to form a nucleoside. The addition of a phosphate group to the nucleoside produces a nucleotide unit.

The typical strand of DNA in a cell consists of 3-6 billion nucleotides, consequently the need for nucleotides for cell replication is significant. To produce sufficient numbers of nucleotides for use in a cell, nucleotide synthesis takes place in a cell through a de novo synthesis pathway and a salvage pathway. The de novo synthesis pathways generally described above have substantial energy requirements. For example, the de novo synthesis of the purine nucleotides requires fourteen biochemical steps.

In the past, because of the presence of de novo and salvage pathways, it was thought that the body could produce all of the nucleotides to meet the physiological demands of the body and therefore nucleotides were not considered to be essential nutrients. However, under conditions of rapid growth or metabolic stress in the body, the demand for nucleotides exceeds the capacity of the de novo synthesis system. Current research also suggests that even though the body is able to recycle nucleotides, the salvage pathways become less effective during times of stress on the body. Consequently, there is an observed reduction in the replication and maturation of rapidly growing cells such as macrophages, lymphocytes and gastrointestinal mucosa during times of stress. Therefore, nucleotides can be considered essential nutrients in certain situations.

Furthermore, it has been observed that bone marrow cells, lymphocytes and erythrocytes that are important to the immune system are not able to synthesize purines. Other tissues such as intestinal mucosa cells and some brain cells are not able to synthesize sufficient quantities of purines to cover their needs. Accordingly, an exogeneous supplementation of nucleotides would be beneficial to these types of cells and associated systems.

### BRIEF SUMMARY OF THE INVENTION

The invention pertains generally to a dietary supplement of concentrated nucleotides and preferably a brewers yeast carrier. The nucleotide supplement may also include antioxidants, amino acids, vitamins and other material that can act in concert with the nucleotides to provide a therapeutic effect on the body. Nucleotides are produced in the body through a de novo pathway that is a time and energy consuming process. The body also has a salvage pathway that permits the uptake of nucleotides from the intestines saving time and energy. However, cells such as bone marrow cells, lymphocytes and erythrocytes that are not able to produce certain nucleotides, benefit from nucleotide supplementation. Consequently, the immune response is quicker and more precise when the cells are supplied with sufficient supplies of nucleotides.

The invention also pertains to the beneficial use of nucleotides in the treatment of certain symptoms and conditions of the body. Generally, dietary supplementation with nucleotides reduces the energy requirement of de novo synthesis of nucleotides. In the intestine, nucleotide supplementation supports the intestinal cells and accelerates healing. Supplementation with nucleosides also improves the state of intestinal flora. Nucleotide supplementation has also been shown to promote recovery from stress and healing in the liver. Dietary supplementation with nucleotides may also have a positive effect on lipoprotein metabolism by increasing plasma HDL levels and decreasing LDL levels. The immune system is further positively stimulated by nucleotide supplementation. An increase to the resistance to bacterial and viral infections is observed along with improved antibody production.

According to one aspect of the invention, a method is provided for the modulation of the HDL and LDL levels in the plasma through the administration of a therapeutic dose of nucleotides to the patient. One particularly beneficial dose is the daily consumption of approximately 1000 milligrams to approximately 2000 milligrams of nucleotide supplement. A second beneficial dose of nucleotides is a daily dose of approximately 1750 to approximately 2000 milligrams for sixty days followed by a daily maintenance dose of approximately 1000 to approximately 1250 milligrams.

According to another aspect of the invention, a method is provided for the treatment of chronic diarrhea and irritable bowel syndrome by administering a therapeutic dose of nucleotides to the digestive system. A dose of approximately 1000 milligrams to approximately 2000 milligrams of nucleotide per day is one beneficial dose. A second therapeutic dose is distributed over three periods. A daily dose of approximately 1500 milligrams to 3000 milligrams is taken for a first fourteen-day period. A daily dose of approximately 1000 milligrams to 3000 milligrams is taken for a second fourteen-day period. A daily dose of approximately 500 milligrams to 1000 milligrams is taken for a thirty-day period.

According to still another aspect of the invention, a method is provided for the treatment cold or flu symptoms by administering a therapeutic dose of nucleotides at the onset of symptoms. One beneficial dose of nucleotides is the administration of approximately 2000 milligrams per day for a fourteen-day course even if the symptoms do not persist.

Notwithstanding the particular benefit of this dosing range for each of the aforementioned methods, it is also believed that higher doses delivered orally would be safe. Titration is a further mechanism believed to provide the ability to test for tolerance to higher doses as necessary to alleviate symptoms.

An object of the invention is to provide a dietary supplement that will efficiently deliver concentrated nucleotides to the body of a patient.

Another object of the invention is to provide a dietary supplement that provides nucleotides that may be combined with an antioxidant, amino acids or vitamins to optimize efficacy.

Another object of the invention is to provide a method of treatment of intestinal disorders using a therapeutic dose of nucleotides to promote healing and intestinal flora.

Yet another object of the invention is to provide a method of modulating cholesterol levels in the body using a dietary supplement.

Another object of the invention is to provide a dietary supplement that can be manufactured easily and at low cost.

Further objects and advantages of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which is for illustrative purposes only:

FIG. 1 is a bar chart depicting the plasma cholesterol concentration (Mean±SD) in the experimental, placebo and control groups during the 60-day trial period.

FIG. 2 is a bar chart depicting the plasma HDL cholesterol concentration (Mean±SD) in the experimental, placebo and control groups during the 60-day trial period.

FIG. 3 is a bar chart depicting the percentage of plasma HDL cholesterol as part of the total cholesterol concentration in the experimental, placebo and control groups during the 60-day trial period.

FIG. 4 is a bar chart depicting the salivary IgA levels (Mean±SD) before and after exercise and pre and post nucleotide supplementation.

FIG. 5 is a bar chart depicting the cortisol levels (Mean±SD) before and after exercise and pre and post nucleotide supplementation.

### DETAILED DESCRIPTION OF THE INVENTION

The present invention provides a range of compositions for dietary supplements enriched with concentrated nucleotides or mixes of nucleotides with nucleosides and other nucleic acid precursors, antioxidants, amino acids, vitamins and the like as well as methods for treatment with such supplements. Such mixes may be particularly adapted to the treatment of a particular medical condition. It will be appreciated that the nucleotide composition may vary as to components and quantities, and that the methods of use may vary as to the specific steps and sequence, without departing from the basic concepts as disclosed herein.

It is to be appreciated that the terms “agent,” “supplement,” and “therapeutic dose” are frequently used throughout this disclosure for the purpose of explaining various aspects of the several embodiments. The term “agent” may represent many different types of materials, including fluids, but may be otherwise such as powders, gels, suspensions, etc. In general, however, “agent” is intended to mean a material that when delivered has a generally useful effect in providing therapy. The term “agent” is also generally used to describe regulated materials having the particular bioactivity of consequence to host organisms or their tissues. The term “supplement” or “agent” is also intended to encompass compounds that, under physiological conditions, are converted into the therapeutically active agents. For example, selected moieties of an agent may be hydrolyzed under physiological conditions to provide the desired molecule. Alternatively, the agent or supplement may be converted by the enzymatic activity of the body.

The term “therapeutic dose” as used herein means that amount of a compound; material, drug or composition that is effective for producing some desired therapeutic effect either systemically or to specific organs or tissues of the body. In general, a suitable dose will be that amount of the compound that is the lowest dose effective to produce a therapeutic effect. Such an effective dose will generally depend upon the factors described above. In addition, the effective dose of the active compound may be administered as two, three, four, five, six or more sub-doses administered separately at appropriate intervals throughout the course of treatment preferably in unit dosage forms. In addition, a dose may mean a total quantity of a therapeutic agent administered over the course of a treatment. The term “stimulate,” refers to any increase, enhancement or augmentation of a physiological system or condition following consumption of the agent or supplement. The term “treatment” is intended to encompass prophylaxis, as well as local or systemic therapy or cure.

It will also be appreciated that the various embodiments of the present invention relate to formulations of a dietary supplement containing a therapeutically effective quantity of nucleotides alone or together with a pharmaceutically acceptable carrier. In particularly preferred embodiment, the nucleotides are concentrated and administered in an inactivated yeast carrier.

The nucleotides of the present invention may have a deoxyribose sugar moiety, a ribose sugar moiety or a combination or deoxyribose and ribose sugar moieties. The nucleotides are preferably in the form of single monomeric units but may also be in the form of oligonucleotides that are broken down into single units (i.e. AMP, GMP, CMP, UMP) in the body. Oligonucleotides of two to six units in length are preferred. However, oligonucleotides of greater lengths may also be suitable. A combination of nucleotides and oligonucleotides of varying lengths may be provided in combination to provide a sustained quantity of nucleotides in the system.

In one embodiment of the invention, nucleotide precursors are included with the concentrated nucleotides to supplement both the de novo and salvage pathways for nucleotides within the cells of the body. Such nucleotide precursors may include purines, pyrimidines, nucleosides, amino acids such as glutamic acid, glycine or asparaginic acid or other intermediary molecules involved in the de novo synthesis pathways.

The preferred carrier is brewers yeast or bakers yeast. However, Imulin or other sugar components and polymethoxylated flavones (PMF's) and tocotrienols may also be beneficially used as carriers.

The dietary supplement of the invention may also include optional agents that improve the therapeutic effect of nucleotides on a particular condition or symptom. Additional agents may also act in concert with the nucleotide supplement to multiply the overall therapeutic effect of the supplement or may provide a synergistic effect. Other additives may stimulate interrelated physiological systems as well as separate systems to provide relief of specific symptoms.

In one embodiment, the dietary supplement includes an antioxidant in addition to the nucleotides. Preferred antioxidants include Vitamin C, Vitamin E, n-acetylcysteine, Pantothenic Acid and Selenium. While these antioxidants are generally preferred, it will be understood that other antioxidants known in the art may also be used alone or in combination and may be selected based on a particular condition or symptom targeted for treatment.

In another embodiment, the dietary supplement includes one or more biologically active amino acids or a salt, or digest thereof, in combination with the nucleotides as active ingredients in the supplement. Supplementation of certain amino acids and similar agents has been found to be beneficial to supporting the de novo nucleotide synthesis and salvage pathways. Amino acid supplementation may also support physiological systems that may have deficiencies due to an ailment, an infection or other stress upon the body. In addition to the amino acids that support the nucleotide production pathways or are considered essential, L-Methionine, L-Arginine, L-Glutamine and L-Lysine are particularly preferred for general applications. Additional amino acids can be included to target a known deficiency or selected to provide a known stimulatory or other effect on the body.

In another embodiment, vitamins, minerals, therapeutic herbal extracts and other nutrients may be added to the nucleotide formulation. Agents such as quercetin, for example, may be selected because of their antioxidant and anti-inflammatory properties. Combinations of vitamins, minerals and other agents may be selected to address targeted symptoms.

In a particularly preferred embodiment, a dietary supplement is provided that has a quantity of concentrated nucleotides in combination with Methionine, Lysine, Inositol, Vitamin C, Vitamin E, Vitamin B12, Folic Acid, Panthothenic Acid and Biotin. This base formulation has been shown to be particularly effective in the treatment of intestinal disorders, compromised immunity and for promoting healing.

The dietary supplements for animals or humans of the present invention may be prepared for administration in a variety of forms that include liquid, powder, capsule or solid pill forms. Nucleotides can be administered alone or as a mixture with other therapeutic compositions as well as a pharmaceutically acceptable carrier or binders. Tablet or pill forms of the supplements may be prepared by methods routine in the art at a variety of dosages. Likewise, the nucleotide preparation of the invention may be administered in the form of elixirs or suspensions containing flavoring or coloring agents. Such liquid preparations may be prepared with pharmaceutically acceptable additives such as suspending agents known in the art. Although not preferred, injectable forms of the concentrated nucleotide preparation of the invention could be prepared by solubilizing the nucleotides in a pharmaceutically acceptable delivery liquid. Under certain conditions nucleotides could also be administered topically in liquid or cream or lotion forms as well as delivered by inhalation.

Various medical conditions and indications may be suitable environments for using the various dietary supplement embodiments disclosed above. The present invention has been shown to be particularly beneficial when applied to the treatment of cholesterol levels, chronic diarrhea or irritable bowel syndrome and immune system deficiencies.

Many physical ailments such as coronary heart disease can be linked to high levels of serum cholesterol. Atherosclerosis, for example, is a disease that is characterized by the progressive accumulation of cholesterol within the walls of the arteries and is one of the leading causes of death in the United States. There is evidence to suggest that lipids that are deposited in atherosclerotic lesions in arterial walls are derived primarily from low-density lipoproteins (LDL) in the blood. Accordingly, a high concentration of LDL in plasma creates a risk of coronary heart disease. In contrast, high concentrations of high-density lipoproteins (HDL) in plasma are considered to be beneficial. It is believed that LDL generally functions to deliver cholesterol from the liver to other tissues of the body and that HDL scavenges tissue cholesterol and transports it back to the liver for destruction. Research suggests that high levels of HDL may be protective of the heart and may also cause regression of existing atherosclerotic plaques. Therefore, the ratio of HDL to LDL should optimally favor comparatively high HDL levels and Low LDL levels for good health.

Current treatments that are available for lowering serum cholesterol have limitations and side effects that make such treatments ineffective or unavailable for some patients. Therefore, there exists a real clinical need in the interventional setting for modulating the ratio of HDL to LDL and total cholesterol to mitigate the risk of the onset of coronary heart disease.

Presented herein is a method for altering the HDL/LDL ratio by increasing the levels of HDL and decreasing the overall levels of all cholesterol by the use of nucleotides. One study disclosed an increase in HDL levels of greater than 40% with sixty days of supplementation.

In one embodiment of the method for the treatment of cholesterol, a therapeutically effective daily dose of nucleotides for an adult is approximately 1000 milligrams to approximately 2000 milligrams administered daily for a course of at least 60 days has been shown to be beneficial. This dose has been effective in decreasing the overall cholesterol concentration in the blood of the patient while increasing the concentration of HDL and improving the HDL/LDL ratio of the patient. A particularly preferred course of treatment includes first course of a daily doses of approximately 1750 milligrams to approximately 2000 milligrams administered for approximately sixty days. If symptoms have not improved sufficiently then additional daily doses can be taken until the symptoms improve satisfactorily. A maintenance dose of approximately 1000 milligrams can be administered daily to maintain the improved ratio of HDL/LDL in the blood.

In another embodiment, the base formulation had a therapeutic amount of niacin or nicotinic acid added to the supplement. Niacin has been shown to reduce production of LDL and concurrently lower LDL levels through a different mechanism than the nucleotide supplement. Other additives may also be included to the base formulation.

Another beneficial use of the nucleotide supplement is in the digestive system and treatment of digestive system ailments such as chronic diarrhea and irritable bowel syndrome. Irritable bowel syndrome (IBS) is a disorder of the function of the intestinal muscles characterized by intermittent abdominal cramps and constipation with alternating periods of diarrhea. Women are affected by IBS about twice as often as men. The exact cause or causes of the syndrome is not well understood although it appears to be due in part to an increased sensitivity of the bowel. Chronic diarrhea and IBS can lead to injury to the interior lining of the intestines that requires repair. Experimental evidence has shown that nucleotide supplementation reduces the recovery time of the cells of intestines of subjects with chronic diarrhea.

For curative or prophylactic treatment of irritable bowel syndrome and chronic diarrhea described above, the oral administration of dosages of a composition of nucleotides is preferred. In practice a health care practitioner may determine the actual dosing regimen, which will be the most suitable for an individual patient, and it will vary with the age, weight and response of the particular patient. A therapeutically effective daily dose of nucleotides will generally be in the range of approximately 1000 milligrams to approximately 2000 milligrams administered daily for an average adult patient with a weight of about 70 kg. For children from the ages of 6 to 12, the typical therapeutically effective daily dose of nucleotides will generally be within the range of approximately 500 milligrams to approximately 1000 milligrams of nucleotides administered daily. It will be seen that the daily dose may be administered in single or multiple doses, once or several times per day. It is preferred that the digestive tract of the patient has a relatively constant exposure to nucleotides. Consequently, a course of multiple sub-doses of nucleotides administered daily is preferred.

In one embodiment, the course of treatment with nucleotides includes a fourteen-day course of daily nucleotide doses of approximately 1500 milligrams to approximately 3000 milligrams. A second fourteen-day course of daily doses of approximately 1000 milligrams to approximately 3000 milligrams follows the first course of treatment. Thereafter, a third course of daily doses of approximately 500 milligrams to approximately 1000 milligrams is administered for approximately thirty days. If necessary, a maintenance dose of approximately 250 milligrams to approximately 500 milligrams of nucleotides can be administered daily to prevent future symptoms.

Experimental evidence has also shown that the fecal flora of patients with irritable bowel syndrome is different than with normal asymptomatic patients. It has been seen that nucleotide supplements restores normal fecal flora and has beneficial effects on the intestine, liver and lipid metabolism and bolsters immunity. Therefore, supplementation with dietary nucleotides may alter symptoms of irritable bowel syndrome or chronic diarrhea by two mechanisms, (1) a direct beneficial effect on the gastrointestinal tract and (2) by restoring normal fecal flora.

The base formulation of the present invention may optionally be augmented with anti-inflammatory agents such as quercetin or the like or muscle relaxants that have been shown to be beneficial in treating acute symptoms of diarrhea.

There is a clear correlation between the nutritional status of an individual and immunocompetency. Exposure to disease causing agents such as viruses and bacteria does not always result in illness and the ultimate determining factor is the condition of the immune system of the individual. Recent studies have shown that dietary supplementation of nucleotides are beneficial to the proper cell-mediated responses to antigen stimulation. Dietary supplementation of nucleotides has been shown to affect a number of immune functions including promoting T-cell maturation and function, enhancing the activity of natural killer cells and aiding in resistance to infection. Nucleotide supplementation has consistently shown to diminish the subjective symptoms and duration of the common cold as well as influenza.

For the treatment and accelerated recovery of the common cold or influenza, one embodiment of the invention provides an adult dose recommendation of approximately 2000 milligrams per day for a course of not less than fourteen days. In one embodiment the daily dose is preferably approximately 500 milligrams of nucleotides administered four times a day.

In another embodiment directed to acute cold and flu symptoms, an initial dose of approximately 2000 milligrams is provided followed by three 1000 milligram doses administered every four hours from the initial dose. Thereafter, approximately 1000 milligram doses four times daily for about fourteen days have been shown to be beneficial. In another embodiment, optional maintenance doses of 500 milligrams per day are provided to bolster the immune system as a prophylactic measure.

It will be understood that the dosages described herein are exemplary of the average case and that there can be individual instances in which higher or lower dosage ranges may be appropriate and such dosage ranges are within the scope of this invention. Titration is a further mechanism believed to provide the ability to test for tolerance to higher doses as necessary to alleviate symptoms.

The present invention may be more particularly described in the following examples that are intended for illustrative purposes only, since numerous modifications, adaptations and variations will be apparent to those skilled in the art.

### EXAMPLE 1

In order to determine the effects of a nucleotide supplementation on the plasma cholesterol and HDL-Cholesterol concentration of the body, thirty young male subjects were randomly assigned to one of three groups, experimental (E), placebo (P) and control (C). The control subjects took no supplement while the experimental group ingested 1.8 g of a nucleotide supplement in three equal doses on a daily basis. The Placebo group ingested 1.8 g daily of an inert substance. The Experimental and Placebo group supplements were identical in nature. The supplementation period lasted 60 days and subjects reported every 15 days after day 0 for blood testing. On each blood collection day, 5 ml of blood was acquired and analyzed for both Cholesterol and HDL-Cholesterol using a commercially available technique.

Data was analyzed with a repeated measures analysis of variance. Where a significant difference was found, further analysis with Fisher's PLSD post-hoc tests was undertaken to determine the significant comparisons. A significance level of p<0.05 was set before the analysis.

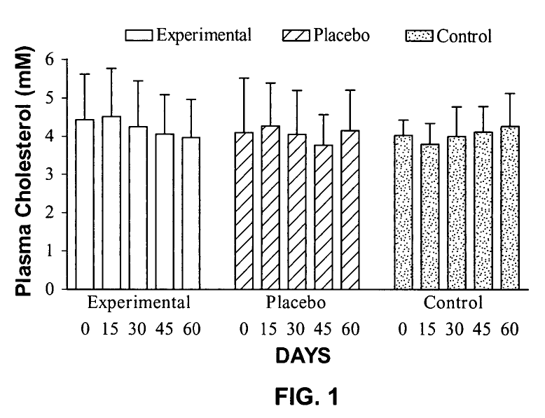

Turning now to FIG. 1, the plasma cholesterol concentration (Mean±SD) in the three groups during the 60-day trial period is shown. The repeated measures ANOVA undertaken on the total Cholesterol data indicated that there was no significant Time * Group interaction (F_{(8,08)}=0.97, p>0.46, Power=0.43) and neither was there any significant group (F_{(2,27)}=0.20, p>0.81, Power=0.10) or time effect (F_{(4,108)}=0.46, p>0.76, Power=0.15) effect.

It can be seen in FIG. 1 that there is a steady decrease in concentration of Cholesterol in the experimental trial results and a regression analysis based on this data was calculated. The equation Y=4.523−0.9*X, where Y=cholesterol concentration and X=days on the nucleotide supplement. On the basis of 100 days on the supplement it was calculated that the total cholesterol concentration would decrease to 3.623 mM.

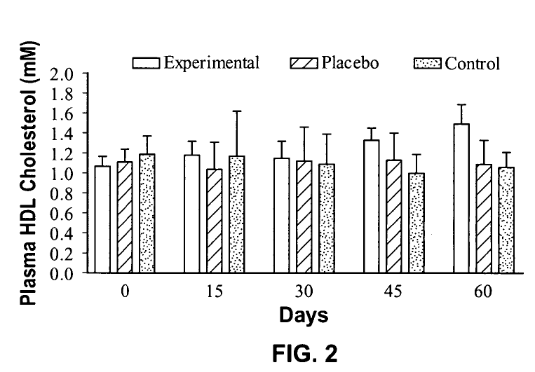

In FIG. 2, the Plasma HDL Cholesterol concentration (Mean±SD) in the three groups during the 60-day trial period is shown. Repeated measures ANOVA conducted on the HDL-Cholesterol data indicated that here was a significant Time * Group interaction (F_{(8,108)}=5.10, p<0.0001, Power=0.99), but no Main effect of either Time (F_{(4,108)}=1.49, p>0.21, Power=0.44), or Group (F_{(2,27})=2.28, p>0.12, Power=0.41).

The ANOVA undertaken on the experimental data indicated that the data was significantly different (F_{(4,45)}=12.70, p<0.0001, Power=1.00). The Fisher's PLSD post hoc analysis indicated that day 0 versus 45 and 60, 15 versus 45 and 60, days 30 versus 45 and 60 and day 45 versus 60 were all significantly (p<0.05) different from each other. There were no significant differences between the placebo (F_{(4,45)}=0.19, p>0.94, Power=0.10) or control groups (F_{(4,45)}=0.83, p>0.51, Power=0.20).

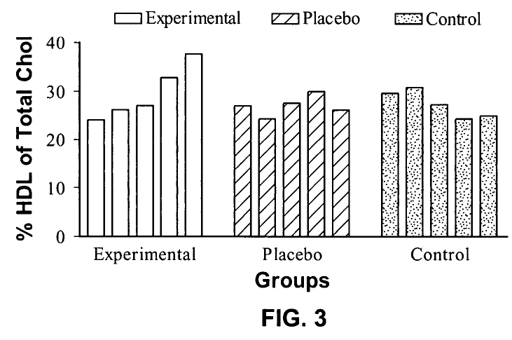

In FIG. 3, the HDL-Cholesterol data is represented as a percentage of the total Cholesterol concentration for the three groups during the 60-day trial is shown. It can be seen that the levels of HDL cholesterol substantially increased in the plasma over the course of the trial. It can also be seen that ratio of HDL to total cholesterol increases while the total levels of all cholesterol decreased in the experimental subjects over the course of the trial.

### EXAMPLE 2

To determine the effects of a nucleotide supplementation on the symptoms of irritable bowel syndrome (IBS) and chronic diarrhea, fifteen eligible subjects presenting with irritable bowel syndrome were selected for the study. The subjects were asked to record a daily symptom score for two weeks before taking nucleotide supplements. The test subjects completed a quality of life questionnaire designed to produce relevant medical information before and after the course of nucleotide supplementation. The test subjects continued to keep a daily symptom diary during the course of supplementation and any adverse effects or tolerability to the supplement were reported.

Subjects took two capsules of supplement three times per day for a period of six weeks. Each capsule contained 500 milligrams of RNA nucleotides and included, nucleotide precursors, Vitamin C, Vitamin B12, Biotin and Folic Acid.

It was observed that the IBS symptom scores and quality of life measures were significantly altered in patients suffering from diarrhea predominant irritable bowel syndrome after six weeks of nucleotide supplementation. After the course of supplementation with nucleotides, a substantial majority of the test subjects observed a reduced severity, frequency and duration of symptoms.

### EXAMPLE 3

To demonstrate the effects of a nucleotide supplementation on the length and severity of certain specific symptoms of cold or flu infection, patients with existing symptoms were given a placebo or a nucleotide supplement and evaluated.

Male and female adults between the ages of 18 and 55 that had developed the first symptoms within 24 hours were seen by a physician to confirm the symptoms. Eligible subjects had a minimum of two of the following symptoms present: cough, headache, hoarseness, muscle ache, nasal drainage, nasal congestion, scratchy throat, sore throat, sneezing and fever.

Three hundred subjects were selected for the study and randomly divided into two groups assigned for nucleotide treatment and placebo. The duration of the study was fourteen days from the onset of symptoms. On day one the subjects took four capsules followed by two capsules every four hours for twelve hours. From day two through day fourteen, the subjects took two capsules four times daily. The participants took all of the capsules through the course of the fourteen-day trial even if their symptoms disappeared.

Blood samples were taken from each subject on days 1, 3, 5 and 7 of the study. Plasma separated from the blood samples was analyzed for total leukocyte count, differential white cell count and lymphocyte subpopulations to assess immune function of the individual subjects. The first blood analysis confirmed the presence of infection. Each participant also kept a diary of symptoms.

Nearly all of the subjects that received a therapeutic dose of nucleotides initiated immediately upon recognition of symptoms reported significantly less subjective symptoms and substantially accelerated recovery than experienced by the subjects taking the placebo. In addition, levels of serum IgA, leukocytes and neutrophil were elevated in the subjects receiving the supplement indicating the strength of the immune response was greater in these subjects over those receiving the placebo.

It was seen that dietary supplementation with nucleotides can reduce the severity of specific symptoms, secondary infections and healing time after natural, non-induced infection by a cold or influenza virus.

### EXAMPLE 4

The effects of nucleotide supplementation on salivary IgA and cortisol after moderate exercise was evaluated. The major immunoglobulinin found in saliva is IgA, which is known to inhibit the attachment and replication of some microorganisms in humans. This immunoglobulin is part of the mucosal immune system that protects the body from airborne pathogens.

A decrease in salivary IgA has been observed in endurance athletes following endurance exercises such as cycling, running, swimming and cross-country skiing. Salivary IgA levels may remain low for several hours after the exercise. This decrease in IgA levels after exercise is thought to account for the increased incidence of upper respiratory track infections in endurance athletes. It has also been observed that athletes have higher salivary cortisol levels at rest and after a day of training than inactive individuals.

Fourteen moderately trained male subjects that were approximately the same age, height and weight completed two 90 minute ergonomic cycle trials. The first trial was conducted before the subjects received the supplement. The second trial was conducted after the subjects had taken either a nucleotide supplement or a placebo for sixty days. The subjects consumed 1800 milligrams of nucleotide supplement or inert placebo three times daily with every meal for the duration of the sixty-day trial. On the day of the second exercise trial the subjects provided an unstimulated saliva sample before and after the exercise. The saliva samples were evaluated for IgA and cortisol levels.

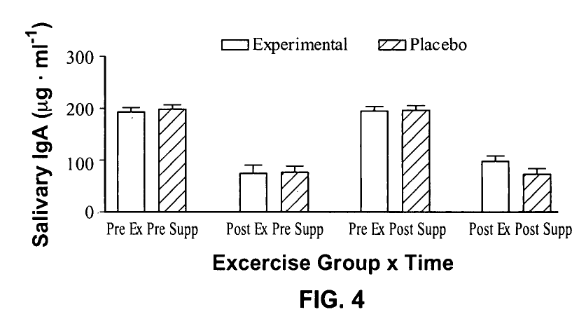

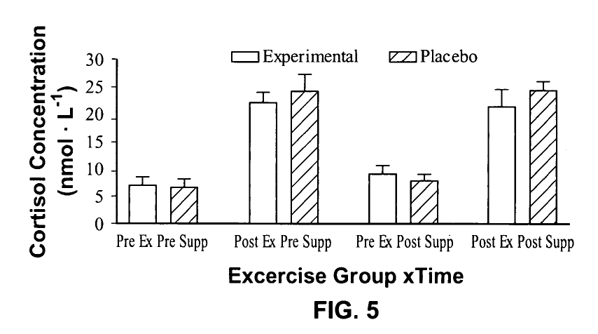

As seen in FIG. 4, there were no significant differences in the salivary IgA levels prior to the supplementation period. However, after supplementation the group receiving the supplement had significantly higher (p<0.01) salivary IgA levels than the group receiving the placebo. Furthermore, the pre-exercise levels of cortisol were the same in both groups before and after supplementation. However, after the supplementation period the post exercise levels of cortisol were significantly lower (p<0.0001) in the group receiving the supplement when compared with the group receiving the placebo as seen in FIG. 5. Accordingly, it can be seen that the group receiving the nucleotide supplements had significantly changed levels of salivary IgA and cortisol demonstrating a positive effect of nucleotide supplementation on the immune function after exercise.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

1. A method for the treatment of diarrhea or irritable bowel syndrome, comprising:
orally administering to a patient in need of treatment a food supplement comprising a therapeutically effective quantity of a nucleotides, together with a pharmaceutically acceptable carrier.

2. A method as recited in claim 1, wherein said therapeutically effective quantity of nucleotides comprises a dose of approximately 1000 milligrams to approximately 2000 milligrams administered daily.

3. A method as recited in claim 1, wherein said therapeutically effective quantity of nucleotides comprises a dose of approximately 500 milligrams to approximately 1000 milligrams administered daily to children from the ages of 6 to 12.

4. A method as recited in claim 1, wherein said therapeutically effective quantity of nucleotides comprises:
a first daily dose of approximately 1500 milligrams to approximately 3000 milligrams administered daily for approximately fourteen days;
a second daily dose of approximately 1000 milligrams to approximately 3000 milligrams administered daily for approximately fourteen days; and
a third daily dose of approximately 500 milligrams to approximately 1000 milligrams administered daily for approximately thirty days.

5. A method as recited in claim 4, further comprising:
a maintenance dose of approximately 250 milligrams to approximately 500 milligrams of nucleotides administered daily.

6. A method as recited in claim 1, wherein said carrier comprises brewers yeast.

7. A method as recited in claim 1, wherein said carrier comprises bakers yeast.

8. A method as recited in claim 1, wherein said carrier comprises a sugar.

9. A method as recited in claim 8, wherein said carrier comprises Imulin.

10. A method as recited in claim 1, wherein said food supplement further comprises at least one quantity of an antioxidant.

11. A method as recited in claim 10, wherein said antioxidant is an antioxidant selected from the group of antioxidants consisting essentially of Vitamin C, Vitamin E, n-acetylcysteine and Selenium.

12. A method as recited in claim 1, wherein said food supplement further comprises at least one quantity of a biologically active amino acid.

13. A method as recited in claim 12, wherein said amino acid is an amino acid selected from the group of amino acids consisting essentially of L-Methonine, L-Lysine, L-Arginine and L-Glutan.

14. A method as recited in claim 1, wherein said food supplement further comprises at least one vitamin selected from the group of vitamins consisting essentially of Vitamin E, Vitamin C, Vitamin B-1, Vitamin B-2, Vitamin B-6, Vitamin B-12, Folic Acid and Biotin.

15. A method as recited in claim 1, wherein said nucleotides are administered in the form of monomers.

16. A method as recited in claim 1, wherein said nucleotides are administered in the form of short chains of nucleotides from approximately two to approximately six units in length.

17. A method as recited in claim 1, wherein said nucleotides comprise ribonucleic acid monomers.

18. A method as recited in claim 1, wherein said nucleotides comprise deoxyribonucleic acid monomers.

19. A method as recited in claim 1, wherein said nucleotides comprise a combination of deoxyribonucleic acid monomers and ribonucleic acid monomers.

20. A method as recited in claim 1, wherein said food supplement further comprises a quantity of nucleosides and d-ribose.

21. A method as recited in claim 1, wherein said food supplement further comprises a quantity of an anti-inflammatory agent.

22. A method as recited in claim 21, wherein said anti-inflammatory agent comprises quercetin.

23. A method for altering the HDL/LDL ratio in the blood of a patient, said method comprising administering to the patient a pharmaceutically effective quantity of nucleotides to alter the ratio of HDL to LDL.

24. A method as recited in claim 23, wherein said therapeutically effective quantity of nucleotides comprises a dose of approximately 1000 milligrams to approximately 2000 milligrams administered daily.

25. A method as recited in claim 23, wherein said therapeutically effective quantity of nucleotides comprises:
a first daily dose of approximately 1750 milligrams to approximately 2000 milligrams administered daily for approximately sixty days;
a second daily dose of approximately 1000 milligrams administered daily to maintain an altered ratio of HDL/LDL in the blood of said patient.

26. A method as recited in claim 23, wherein said carrier comprises brewers yeast.

27. A method as recited in claim 23, wherein said carrier comprises bakers yeast.

28. A method as recited in claim 23, wherein said food supplement further comprises at least one quantity of an antioxidant.

29. A method as recited in claim 28, wherein said antioxidant is an antioxidant selected from the group of antioxidants consisting essentially of Vitamin C, Vitamin E, n-acetylcysteine and Selenium.

30. A method as recited in claim 23, wherein said quantity of nucleotides further comprises at least one quantity of a biologically active amino acid.

31. A method as recited in claim 30, wherein said amino acid is an amino acid selected from the group of amino acids consisting essentially of L-Methonine, L-Lysine, L-Arginine and L-Glutan.

32. A method as recited in claim 23, wherein said food supplement further comprises at least one vitamin selected from the group of vitamins consisting essentially of Vitamin E, Vitamin C, Vitamin B-1, Vitamin B-2, Vitamin B-6, Vitamin B-12, Folic Acid and Biotin.

33. A method as recited in claims 23, wherein said quantity of nucleotides further comprises a systemic LDL reducing agent.

34. A method as recited in claims 33, wherein said systemic LDL reducing agent comprises Inositol.

35. A method as recited in claim 23, wherein said nucleotides are administered in the form of monomers.

36. A method as recited in claim 23, wherein said nucleotides are administered in the form of short chains of nucleotides from approximately two to approximately six units in length.

37. A method as recited in claim 23, wherein said nucleotides comprise ribonucleic acid monomers.

38. A method as recited in claim 23, wherein said nucleotides comprise deoxyribonucleic acid monomers.

39. A method as recited in claim 23, wherein said nucleotides comprise a combination of deoxyribonucleic acid monomers and ribonucleic acid monomers.

40. A method as recited in claim 23, wherein said food supplement further comprises a quantity of nucleosides and d-ribose.

41. A method of treatment of a human patient suffering from low HDL Cholesterol levels, said method comprising administering to said patient an amount of nucleotides sufficient to substantially increase the circulating levels of HDL and substantially decreasing the overall circulating levels of all cholesterol.

42. A method as recited in claim 41, said method further comprising monitoring HDL and LDL levels during a course of treatment with nucleotides.

43. A method for the treating cold or flu symptoms, comprising:
orally administering to a patient in need of treatment a food supplement comprising a therapeutically effective quantity of a nucleotides at the onset of symptoms.

44. A method as recited in claim 43, wherein said therapeutically effective quantity of nucleotides comprises a dose of approximately 2000 milligrams administered daily for approximately fourteen days.

45. A method as recited in claim 43, wherein said therapeutically effective quantity of nucleotides comprises:
a first dose of approximately 2000 milligrams administered at the onset of symptoms;
a second dose of three administrations of approximately 1000 milligrams every four hours from the first dose; and
a third dose of approximately 1000 milligram administered four times daily for approximately fourteen days.

46. A method as recited in claim 41, said method further comprising administering a maintenance dose of approximately 500 milligrams per day.

47. A method as recited in claim 43, wherein said carrier comprises brewers yeast.

48. A method as recited in claim 43, wherein said carrier comprises bakers yeast.

49. A method as recited in claim 43, wherein said food supplement further comprises at least one quantity of an antioxidant.

50. A method as recited in claim 49, wherein said antioxidant is an antioxidant selected from the group of antioxidants consisting essentially of Vitamin C, Vitamin E, n-acetylcysteine and Selenium.

51. A method as recited in claim 43, wherein said quantity of nucleotides further comprises at least one quantity of a biologically active amino acid.

52. A method as recited in claim 51, wherein said amino acid is an amino acid selected from the group of amino acids consisting essentially of L-Methonine, L-Lysine, L-Arginine and L-Glutan.

53. A method as recited in claim 43, wherein said food supplement further comprises at least one vitamin selected from the group of vitamins consisting essentially of Vitamin E, Vitamin C, Vitamin B-1, Vitamin B-2, Vitamin B-6, Vitamin B-12, Folic Acid and Biotin.

54. A pharmaceutical composition, comprising:
a therapeutically effective amount of the nucleotides together with a pharmaceutically acceptable carrier.

55. A pharmaceutical composition as recited in claim 54, wherein said nucleotides are in the form of short chains of nucleotides from approximately two to approximately six units in length;
wherein said nucleotide chains are separated into individual units by the body of said patient.

56. A pharmaceutical composition as recited in claim 54, wherein said nucleotides comprise ribonucleic acid monomers.

57. A pharmaceutical composition as recited in claim 54, wherein said nucleotides comprise deoxyribonucleic acid monomers.

58. A pharmaceutical composition as recited in claim 54, wherein said nucleotides comprise a combination of deoxyribonucleic acid monomers and ribonucleic acid monomers.

59. A pharmaceutical composition as recited in claim 54, wherein said composition further comprises a quantity of nucleosides and d-ribose.

60. A pharmaceutical composition as recited in claim 54, wherein said carrier comprises brewers yeast.

61. A pharmaceutical composition as recited in claim 54, wherein said carrier comprises bakers yeast.

62. A pharmaceutical composition as recited in claim 54, wherein said carrier comprises a sugar.

63. A pharmaceutical composition as recited in claim 62, wherein said carrier comprises Imulin.

64. A pharmaceutical composition as recited in claim 54, wherein said carrier comprises a polymethoxylated flavone (PMF).

65. A pharmaceutical composition as recited in claim 54, wherein said carrier comprises a tocotrienol.

66. A pharmaceutical composition as recited in claim 54, wherein said composition further comprises at least one quantity of an antioxidant.

67. A pharmaceutical composition as recited in claim 66, wherein said antioxidant is an antioxidant selected from the group of antioxidants consisting essentially of Vitamin C, Vitamin E, n-acetylcysteine and Selenium.

68. A pharmaceutical composition as recited in claim 54, wherein said composition further comprises at least one quantity of a biologically active amino acid.

69. A pharmaceutical composition as recited in claim 68, wherein said amino acid is an amino acid selected from the group of amino acids consisting essentially of L-Methonine, L-Lysine, L-Arginine and L-Glutan.

70. A pharmaceutical composition as recited in claim 54, wherein said composition further comprises at least one vitamin selected from the group of vitamins consisting essentially of Vitamin E, Vitamin C, Vitamin B-1, Vitamin B-2, Vitamin B-6, Vitamin B-12, Folic Acid and Biotin.

71. A pharmaceutical composition as recited in claim 54, wherein said composition further comprises a quantity of an anti-inflammatory agent.

72. A pharmaceutical composition as recited in claim 71, wherein said anti-inflammatory agent comprises quercetin.

73. A nutritional supplement, comprising:
a therapeutically effective quantity of nucleotides;
at least one antioxidant;
at least one amino acid; and
at least one vitamin; and
a carrier.

74. A nutritional supplement as recited in claim 73, wherein said antioxidant is at least one antioxidant selected from the group of antioxidants consisting essentially of Vitamin C, Vitamin E, n-acetylcysteine and Selenium.

75. A nutritional supplement as recited in claim 73, wherein said vitamin comprises at least one vitamin selected from the group of vitamins consisting essentially of Vitamin E, Vitamin C, Vitamin B-1, Vitamin B-2, Vitamin B-6, Vitamin B-12, Folic Acid and Biotin.

76. A nutritional supplement as recited in claim 73, wherein said amino acid is an amino acid selected from the group of amino acids consisting essentially of L-Methonine, L-Lysine, L-Arginine and L-Glutan.
